(19) United States
(12) Patent Application Publication
(10) Pub. No.: US 2013/0072657 A1
(43) Pub. Date: Mar. 21, 2013

(54) CELL GROWTH INHIBITOR DERIVED FROM p16INK4a AND METHOD OF SYNTHESIZING THE SAME

(71) Applicants: Najmeh Fahham, TEHRAN (IR); Soroush Sardari, TEHRAN (IR); Mohammad Hossein Ghahremani, TEHRAN (IR)

(72) Inventors: Najmeh Fahham, TEHRAN (IR); Soroush Sardari, TEHRAN (IR); Mohammad Hossein Ghahremani, TEHRAN (IR)

(73) Assignees: Najmeh Fahham, Tehran (IR); Mohammad Hossein Ghahremani, Tehran (IR); PASTEUR INSTITUTE OF IRAN, TEHRAN (IR); Soroush Sardari, Tehran (IR)

(21) Appl. No.: 13/237,116

(22) Filed: Sep. 20, 2011

(51) Int. Cl. C07K 14/00; C12P 21/00

(52) U.S. Cl. 530/300; 435/69.2

(57) ABSTRACT

The various embodiments herein provide a cell growth inhibitor derived from p16^{INK4a} gene comprising a C-terminal segment of the p16^{INK4a} gene, two ankyrin repeats i.e. ankyrin III and ankyrin IV of the p16^{INK4a} gene, two loops i.e. loop 2 and loop 3 of the p16^{INK4a} gene and residues along with the loop 2 consisting Phe⁹⁰, Glu⁸⁸, Gly⁸⁹, Asp⁹² and Asp⁸⁴ residues. The cell growth inhibitor is a truncated form of the p16^{INK4a} gene and encompasses 66-156 amino acids of a full length p16^{INK4a} gene. The cell growth inhibitor has 42% growth inhibition at 24 hrs. The embodiments herein also provide a method of synthesizing the cell growth inhibitor using a polymer chain reaction method.

### BACKGROUND

1. Technical Field

The embodiments herein generally relate to a field of cancer treatment and particularly to tumor suppressor gene. The embodiments herein more particularly relate a cell growth inhibitor derived from p16 tumor suppressor gene. The embodiments herein also relate to a method of synthesizing the cell growth inhibitor from the native p16 gene.

2. Description of the Related Art

A regulation of cell cycle progression in G1-S transition state is tightly controlled by cyclins, cyclin dependent kinases (CDK) and CDK inhibitors. The p16^{INK4a}, a negative regulator of CDK4, is a 156 amino acid protein, comprising of four ankyrin repeats, which are believed to be involved in CDK4 interaction. A tumor suppressor p16 has been found to be inactive in various cancer cells through deletions, missense and nonsense mutations and hypermethylation (>70 different types of cancers to date).

A progression through the different phases of the cell cycle is controlled at several checkpoints, specifically G1-S transition state. A genetic analysis has revealed that certain proteins involved in this phase of the cell cycle have most often altered in human tumors. In particular, the cyclin-dependent kinase (CDK)-cyclin D/INK4/pRb/E2F cascade has been found to be altered in more than 80% of human neoplasias. This process begins with the activation of the CDK4/6 upon binding to cyclin D with the subsequent phosphorylation of pRb and release of the E2F transcription factor. Upon binding to the G1 kinases, p16 inhibits the phosphorylation of the retinoblastoma (Rb) protein by CDK4 and CDK6, blocks the activity of E2F transcription factors and the expression of genes essential for the onset of S phase and mitosis, which results in arresting the cells in the G1 phase of the cell cycle.

The p16^{INK4a} tumor suppressor gene, located at the chromosomal 9p21 region, is a specific CDK4/6 inhibitor. Since it was first reported in 1994, p16 has been considered as one of the most altered genes in a wide variety of malignant human tumors (>70 different types). It has been inactivated by several molecular mechanisms including homozygous deletions, point mutations and hyper methylation in CpG islands (Xie et al. 2005). These inactivating mutations severely affect the stability of the tertiary structure of p16 and its function.

The p16 comprises mainly of four contiguous ankyrin repeats, linear array of a repeating helix-turn-helix structure which are believed to be involved in protein-protein interactions. Each ankyrin repeat exhibits a helix turn helix structure. The helices are designated as 1, 2, 3, etc. The four H T H motifs are connected by three loops in beta and gamma turn structure. The tertiary structure of p16 has a putative cleft for binding to the non-catalytic side of CDK4/6. According to the random mutagenesis studies on p16 gene, there is a large contact surface between p16 and CDK4, while many amino acids throughout the four ankyrin repeats are important for the interaction. On the other hand, based on the molecular dynamic studies, some interactions between p16 and CDK4/6 are functionally redundant. In consistence with these studies, the deletion of C-terminal portion after codon 135 has been shown to have no effect on the activity of p16 in vitro. Moreover, the tumor-associated mutations in the second, third, and early fourth ankyrin repeats occur more frequently at the residues that are invariant in all members of the p16 family which results in a loss of function; while the mutations in the first and late fourth ankyrin repeats are less likely to disrupt p16 function (Yarbrough, Buckmire et al. 1999).

A detailed structural analysis has shown that the mutations are present with a high frequency in three regions: loop 2, entire ANK III, and from loop 3 to the beginning of helix IVB (Byeon, Li et al. 1998). Regarding the importance of ANK III, it has been proposed that a short 20-residue peptide derived from this ankyrin repeat with the same sequence as fragment 84-103 of p16 can be selected for its ability to bind and inhibit CDK4 in vitro.

Along with the ankyrin repeats that are important motifs in protein-protein interactions, the contribution of the loops to interaction with CDK4/6 cannot be denied although they show less defined structure due to the conformational flexibility in p16.

Hence, there is a need to develop a tumor suppressor gene that does not get altered, remains active in various cancer cells and inhibits cell growth and proliferation equivalent to p16.

The above mentioned shortcomings, disadvantages and problems are addressed herein and which will be understood by reading and studying the following specification.

### OBJECTIVES OF THE EMBODIMENTS HEREIN

The primary object of the embodiments herein is to provide a cell growth inhibitor that inhibits cell growth and proliferation equivalent to p16.

Another object of the embodiments herein is to provide a cell growth inhibitor that interacts with CDK4/6 enzyme, blocks the entry into S phase of the cell cycle and suppresses growth of the tumor cells.

Yet another object of the embodiments herein is to provide a cell growth inhibitor that is used for gene therapy of cancers having non-functional p16 due to various inactivation reasons such as mutations, deletions or epigenetic silencing.

Yet another object of the embodiments herein is to provide a cell growth inhibitor that serves as a template for a drug discovery or is used as a template for peptidomimetic drug design.

Yet another object of the embodiments herein is to provide a cell growth inhibitor that is used alone or in combination with DNA damaging agents like chemotherapy or radiotherapy to increase the efficacy and to decrease the adverse affects.

Yet another object of the embodiments herein is to provide a cell growth inhibitor that is expressed properly and fold independently.

Yet another object of the embodiments herein is to provide a cell growth inhibitor that is capable of inducing alteration in the expression of apoptotic and cell cycle involved proteins such as Mcl-1, Bcl2, p21, Bim, Puma and Noxa.

Yet another object of the embodiments herein is to provide a cell growth inhibitor that functions as good as the full length protein in inhibiting tumor cell proliferation.

Yet another object of the embodiments herein is to provide a method of synthesizing a stable and functional fragment of p16 tumor suppressor.

These and other objects and advantages of the embodiments herein will become readily apparent from the following detailed description taken in conjunction with the accompanying drawings.

### SUMMARY

The various embodiments herein provide a cell growth inhibitor derived from p16^{INK4a} gene comprising a C-terminal segment of the p16^{INK4a} gene, at least two ankyrin repeats, at least two loops and a plurality of residues along with the loop 2. The at least two ankyrin repeats are ankyrin III and ankyrin IV of the p16^{INK4a} gene. The loops are loop 2 and loop 3 of the p16^{INK4a} gene. The plurality of residues includes Phe⁹⁰, Glu⁸⁸, Gly⁸⁹, Asp⁹² and Asp⁸⁴. The plurality of residues interacts and binds with Cyclin-dependent kinase 4 (CDK4) enzymes. The plurality of residues have selectivity towards cyclin-dependent kinase 4/6 (CDK4/6) enzyme. The plurality of residues causes distortion of ATP-binding sites of a cell. The cell growth inhibitor has a gene sequence, wherein the gene sequence is SEQ ID NO. 1. The cell growth inhibitor is a truncated form of the p16^{INK4a} gene. The cell growth inhibitor encompasses 66-156 amino acids of a full length p16^{INK4a} gene. The cell growth inhibitor includes the highest frequency regions considering the inactivating mutations corresponding to the residues 71-76, 80-102 and 107-127. The cell growth inhibitor has 42% growth inhibition at 24 hrs.

According to another embodiment herein, a method for synthesizing a cell growth inhibitor comprises generating a truncated form and cloning the generated truncated form into pcDNA3.1 mammalian expression vector. The truncated form is generated by amplifying an expression constructs containing p16 encoding regions and regulatory elements necessary for the expression of a transcript using a Polymeric Chain Reaction (PCR) using a specific primer pairs carrying restriction enzyme sites. The specific primer pairs are a forward and a reverse primer. The forward primer is GAGGAATTCACCATGCACGGCGC (SEQ ID NO: 2) and the reverse primer is TATGCGGCCGCTCACTTGTCGTC (SEQ ID NO: 3). The cloning is done on the cloning sites. The cloning sites are EcoRI and NotI. The EcoRI is a cloning site for a forward primer. The NotI is a cloning site for a reverse primer. The cell growth inhibitor is a truncated form of the p16^{INK4a} gene. The cell growth inhibitor encompasses 66-156 amino acids of a full length p16^{INK4a} gene. The cell growth inhibitor has 42% growth inhibition at 24 hrs.

According to one embodiment herein, a method is provided for synthesizing a cell growth inhibitor. The method comprising the steps of generating a truncated form, cloning the generated truncated form into pcDNA3.1 mammalian expression vector and obtaining the cell growth inhibitor. The truncated form is generated by amplifying an expression constructs containing p16 encoding regions and regulatory elements necessary for the expression of a transcript using a Polymeric Chain Reaction (PCR) method. The cell growth inhibitor includes a C-terminal segment of the p16^{INK4a} gene, at least two ankyrin repeats, at least two loops and a plurality of residues along with the loop 2. The at least two ankyrin repeats are ankyrin III and ankyrin IV of the p16^{INK4a} gene. The loops are loop 2 and loop 3 of the p16^{INK4a} gene. The plurality of the residues includes Phe⁹⁰, Glu⁸⁸, Gly⁸⁹ Asp⁹² and Asp⁸⁴. The plurality of residues interacts and binds with Cyclin-dependent kinase 4 (CDK4) enzymes. The plurality of residues have a selectivity towards cyclin-dependent kinase 4/6 (CDK4/6) enzyme. The plurality of residues causes distortion of the ATP-binding sites of a cell. The cell growth inhibitor consists of a gene sequence, wherein the gene sequence is SEQ ID NO. 1. The cell growth inhibitor is a truncated form of the p16^{INK4a} gene. The cell growth inhibitor encompasses 66-156 amino acids of a full length p16^{INK4a} gene. The cell growth inhibitor includes the highest frequency regions considering the inactivating mutations corresponding to residues 71-76, 80-102 and 107-127. The cell growth inhibitor has 42% growth inhibition at 24 hrs.

According to an embodiment herein, the cell growth inhibitor structure comprises a truncated form (p16⁶⁶⁻¹⁵⁶) encompassing amino acids 66 to 156 of a full length p16 gene. The truncated form includes a C-terminal segment of a p16 gene comprising ankyrin repeats III and IV along with loops 2 and 3.

According to a preferred embodiment herein, the truncated form G (p16⁶⁶⁻¹⁵⁶) includes ankyrin repeats III, IV along with loops 2 and 3 which posses the most critical amino acids for CDK4/6 interaction. An expression and interaction analysis proposes that this truncated form folds independently and properly into a native like and stable structure. Ankyrin repeat III, including the most important residues for CDK4 interaction, exists in this truncated form.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The other objects, features and advantages will occur to those skilled in the art from the following description of the preferred embodiment and the accompanying drawings in which:

FIG. 1 shows a schematic structural diagram of a cell growth inhibitor according to the embodiments herein.

FIG. 2 shows a gene sequence, SEQ ID NO. 1 for a cell growth inhibitor according to the embodiments herein.

FIG. 3A shows a gel electrophoresis analysis of the generated p16 truncated forms, wherein the PCR amplified p16 truncated forms resolved on 1.5% agarose gel and visualized by ethidium bromide staining, in a cell growth inhibitor according to the embodiments herein.

FIG. 3B shows the gel electrophoresis analysis for the truncated forms cloned into pcDNA3.1 mammalian expression vector and digested with appropriate restriction enzymes, in a cell growth inhibitor according to the embodiments herein.

FIG. 4 shows a graphical representation of the effect of p16 and its four truncated forms on the growth and viability of HT-1080 cell line in a cell growth inhibitor according to the embodiments herein.

FIG. 5A shows a graphical representation of the effect of p16 and the truncated forms on colony formation in HT-1080 cells in a cell growth inhibitor according to the embodiments herein.

FIG. 5B shows a top view of the petridishes showing the growth of colonies when stained with crystal violet in a cell growth inhibitor according to the embodiments herein.

FIG. 6A shows an mRNA expression analysis of p16 and the various truncated forms in a cell growth inhibitor according to the embodiments herein.

FIG. 6B shows a protein expression analysis when evaluated by immunoblot analysis in cells transfected with p16 and the various truncated forms due to a cell growth inhibitor according to the embodiments herein.

FIG. 6C shows an immune fluorescent staining of the transfected cells due to a cell growth inhibitor according to the embodiments herein.

FIG. 7 shows the immune-precipitation analysis showing the protein interaction of p16 full length and p16⁶⁶⁻¹⁵⁶ truncated form with CDK4 and CDK6 in a cell growth inhibitor according to the embodiments herein.

FIG. 8 shows an effect of p16 full length (full) and p16⁶⁶⁻¹⁵⁶ (66-156) truncated from on expression of pro and anti-apoptotic proteins in a cell growth inhibitor according to the embodiments herein.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The various embodiments herein provide a cell growth inhibitor derived from p16^{INK4a} gene comprising a C-terminal segment of the p16^{INK4a} gene, at least two ankyrin repeats, at least two loops and a plurality of residues along with the loop 2. The at least two ankyrin repeats are ankyrin III and ankyrin IV of the p16^{INK4a} gene. The loops are loop 2 and loop 3 of the p16^{INK4a} gene. The plurality of residues includes Phe⁹⁰, Glu⁸⁸, Gly⁸⁹ Asp⁹² and Asp⁸⁴. The plurality of residues interacts and binds with Cyclin-dependent kinase 4 (CDK4) enzymes. The plurality of residues has a selectivity towards cyclin-dependent kinase 4/6 (CDK4/6) enzyme. The plurality of residues causes a distortion of ATP-binding sites of a cell. The cell growth inhibitor has a gene sequence, wherein the gene sequence is SEQ ID NO. 1. The cell growth inhibitor is a truncated form of the p16^{INK4a} gene. The cell growth inhibitor encompasses 66-156 amino acids of a full length p16^{INK4a} gene. The cell growth inhibitor includes the highest frequency regions considering the inactivating mutations corresponding to the residues 71-76, 80-102 and 107-127. The cell growth inhibitor has 42% growth inhibition at 24 hrs.

The truncated form is p16⁶⁶⁻¹⁵⁶. The truncated form in the embodiments herein includes ankyrin repeats III, IV along with loops 2 and 3 that posses the most critical amino acids for CDK4/6 interaction. An expression and interaction analysis proposes that this fragment folds independently and properly into a native like and stable structure. The Ankyrin repeat III, including the most important residues for CDK4 interaction is present in this truncated form. The presence of ANKIV as well as the C-terminal part may influence the stability and conformation of the remainder part of fragment G in a way that causes the proper folding and makes it appropriate for CDK4 binding and inhibition. The loop 2 is not only important for CDK4/6 interaction, but also critical for cell cycle and proliferation inhibition. Along with loop 2, truncated structure or form embraces other critical residues for CDK4 interaction. The residues are Phe⁹⁰, Glu⁸⁸, Gly⁸⁹, Asp⁹² and Asp⁸⁴. These residues have roles in selectivity toward CDK4/6, distortion of the ATP-binding site and binding to CDK4 respectively. Moreover, the truncated form in the embodiments herein includes the highest frequency regions considering the inactivating mutations corresponding to residues 71-76, 80-102 and 107-127.

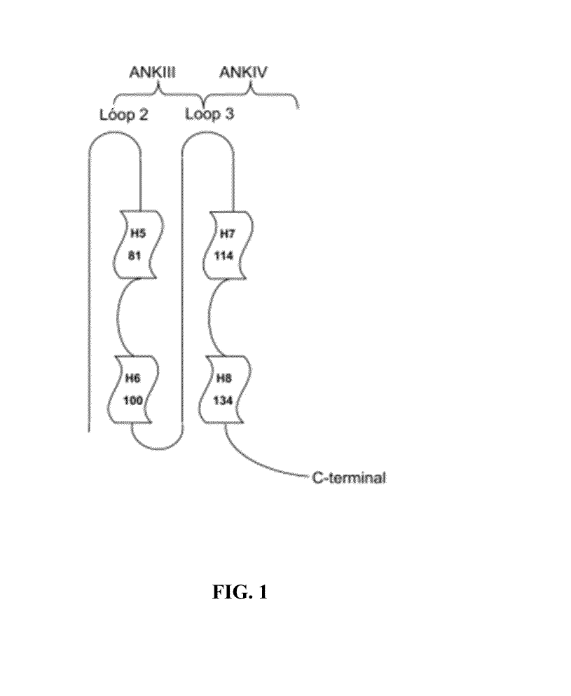

FIG. 1 shows a structural diagram of the cell growth inhibitor according to the embodiments herein. With respect to FIG. 1, the arrangement of the two ankyrin repeats ANKIII and ANKIV, and the loops 2 and 3 with a C terminal can be seen.

In the method of synthesizing the cell growth inhibitor, four truncated forms named E, B, H and G were designed and considered to be characterized according to their mode of effect on growth inhibition of HT-1080 human fibrosarcoma cells. For a generation of the truncated forms, the expression constructs which contain different p16 encoding regions and other regulatory elements necessary for the expression of a transcript were amplified by PCR and cloned into pcDNA3.1 mammalian expression vector. The G construct was prepared carrying a flag tag at its C-terminus. In designing and selecting the truncated forms, it was taken into consideration that although the conserved ANK motifs are important for CDK4 interaction, the intervening loops are also critical in a way that the removal could weaken the interaction toward CDK4. To figure out if the truncated forms can inhibit cellular proliferation, they were introduced to HT-1080 fibrosarcoma cells, lacking functional p16, by transfection using FuGENE® 6 transfection reagent. The cells were analysed 24-48 h after a transfection for expression analysis, a growth suppression, cell cycle and other effects on the cell.

### Experimental Data

To uncover the functional domain of p16, the different truncated structures (including different ankyrin motifs and loops) were generated based on in silico screening according to their interaction with CDK4. The effect of the generated truncated structures on growth inhibition and cell cycle were then evaluated in HT-1080, p16-deficient and wild type pRB fibrosarcoma cell line. The results reported here indicate that p16 C-terminal half including ANK III and IV together with loop 2 and 3 can efficiently behave similar to the full length p16, providing a functional domain as a therapeutic target for blocking CDK4/6 in cancer cells.

Cell Lines and Culture Conditions:

The human fibrosarcoma cell line, HT-1080 (p16 null) (National Cell Bank of Iran, Pasteur Institite of Iran) was maintained in RPMI-1640 medium (BioSera, UK) supplemented with 10% fetal bovine serum, 100 units/ml penicillin and 100 μg/ml streptomycin (all from Gibco, UK) at 37° C. in humidified incubator containing 5% CO₂.

PCR and Plasmids Construction:

The human p16 wild type gene was purchased from Genecopoeia (USA). Four truncated constructs, named p16¹⁻⁸⁰, p16⁶⁶⁻¹¹⁴, p16⁶⁶⁻¹⁵⁶ and p16⁸⁰⁻¹⁵⁶ were generated. The truncated forms were generated by PCR using specific primer pairs carrying restriction enzyme sites and cloned into pcDNA3.1 mammalian expression vector.

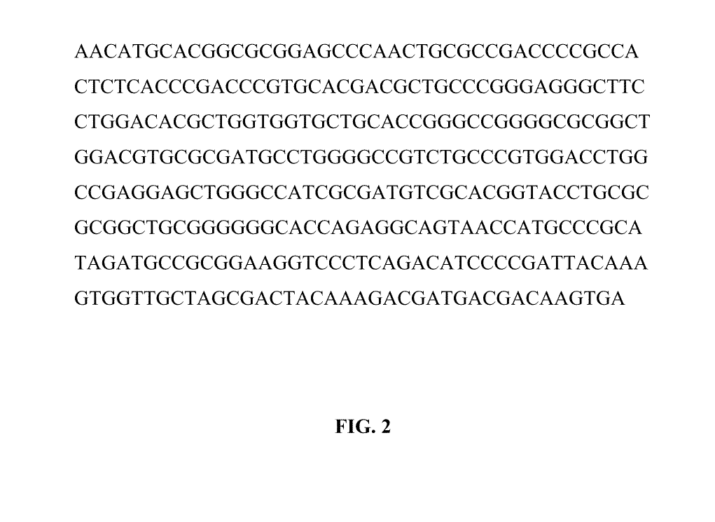

FIG. 2 shows a gene sequence, SEQ ID NO. 1 for the cell growth inhibitor according to the embodiments herein. The cell growth inhibitor has a gene sequence, wherein the gene sequence is SEQ ID NO. 1. a cell growth inhibitor comprises generating a truncated form and cloning the generated truncated form into pcDNA3.1 mammalian expression vector. The truncated form is generated by amplifying expression constructs containing p16 encoding regions and regulatory elements necessary for the expression of a transcript using Polymeric Chain Reaction (PCR) using specific primer pairs carrying restriction enzyme sites. The specific primer pairs are a forward and a reverse primer. The forward primer is GAGGAATTCACCATGCACGGCGC (SEQ ID NO: 2) and the reverse primer is TATGCGGCCGCTCACTTGTCGTC (SEQ ID NO: 3). The cloning is done on cloning sites. The cloning site are EcoRI and NotI. The EcoRI is a cloning site for a forward primer. The NotI is a cloning site for a reverse primer. The cell growth inhibitor is a truncated form of the p16^{INK4a} gene. The cell growth inhibitor encompasses 66-156 amino acids of a full length p16^{INK4a} gene. The cell growth inhibitor has 42% growth inhibition at 24 hrs.

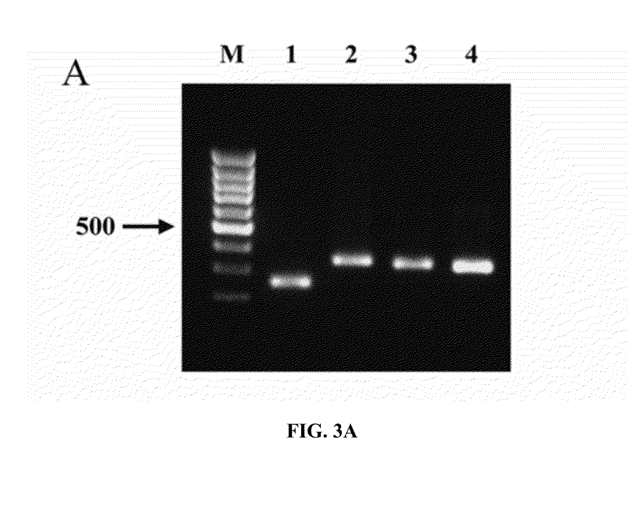

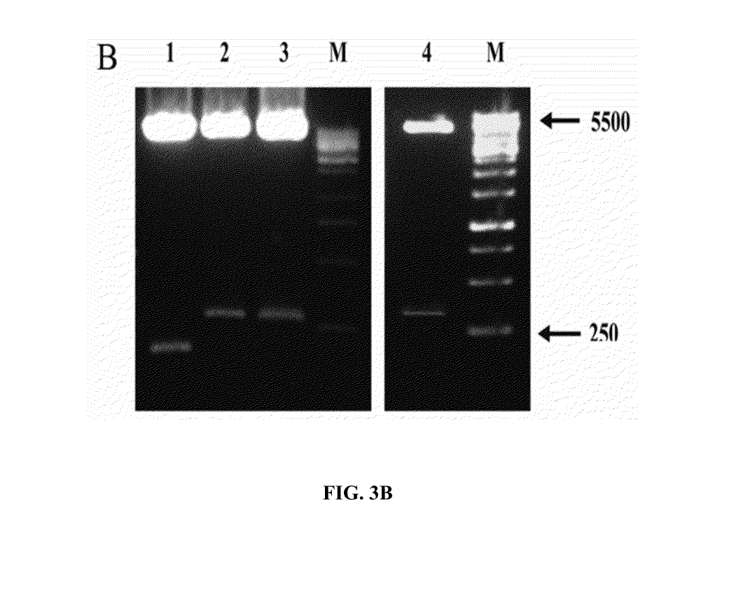

FIG. 3A shows the gel electrophoresis analysis of the generated p16 truncated forms, wherein the PCR amplified p16 truncated forms resolved on 1.5% agarose gel and visualized by ethidium bromide staining. With respect to FIG. 3A, the lane 1 shows the bands for p16⁶⁶⁻¹¹⁴ truncated form (247 bp), lane 2 shows the bands for p16¹⁻⁸⁰ truncated form (345 bp), lane 3 shows the band for p16⁸⁰⁻¹⁵⁶ truncated form (322 bp), lane 4 shows the band for p16⁶⁶⁻¹⁵⁶ truncated form (327 bp) and DNA ladder is represented by M.

FIG. 3B shows the gel electrophoresis analysis for the truncated forms cloned into pcDNA3.1 mammalian expression vector and digested with appropriate restriction enzymes. With respect to FIG. 3B, the lane 1 shows the bands for p16⁶⁶⁻¹¹⁴ truncated form (247 bp), lane 2 shows the bands for p16¹⁻⁸⁰ truncated form (345 bp), lane 3 shows the band for p16⁸⁰⁻¹⁵⁶ truncated form (322 bp), lane 4 shows the band for p16⁶⁶⁻¹⁵⁶ truncated form (327 bp) and DNA ladder is represented by M. TABLE 1 shows the primers and cloning sites used to construct p16 truncated forms. For proper expression, the ATG, Kozak sequence and the stop codon were engineered in the constructs.

TABLE 1 showing the primers and cloning sites used to construct p16 truncated forms

| | SEQ | | | Product |
|---|---|---|---|---|
| | ID | | Cloning | size |
| Construct | NO | Primer pairs | site | (bp) |
| B | 4 | Forward: | KpnI | 345 |
| | | TTGAAGGAATTCGGTACC | XhoI | |
| | | ATGGAGCCG | | |
| | 5 | Reverse: | | |
| | | CGTCGTGCTCGAGTCGGG | | |
| | | TGAGAGTG | | |
| E | 6 | Forward: | HindIII | 247 |
| | | GGCGGAGAAGCTTGCGAT | XhoI | |
| | | GCACG | | |
| | 7 | Reverse: | | |
| | | CCAGCTCGAGGGGCAGAC | | |
| | | GGC | | |
| G | 2 | Forward: | EcoRI | 327 |
| | | GAGGAATTCACCATGCAC | NotI | |
| | | GGCGC | | |
| | 3 | Reverse: | | |
| | | TATGCGGCCGCTCACTTG | | |
| | | TCGTC | | |
| H | 8 | Forward: | HindIII | 322 |
| | | CGCCAAGCTTACCATGCGA | XbaI | |
| | | CCCGTGC | | |
| | 9 | Reverse: | | |
| | | CCTCTAGAATCGGGGATGT | | |
| | | CTGAGGG | | |

The truncated forms were amplified in a 50 μl reaction mixture containing 100 ng p16 gene, 10 pmol of each primer, 0.2 mM dNTP, 1.5 mM MgCl₂, 0.5 U Taq polymerase, 1×PCR buffer (all from CinnaGen, Iran) and 5% DMSO using the following touchdown PCR conditions: 10 cycles of 94° C. for 30 s, 65° C.-0.5° C. per cycle for 45 s, 72° C. for 30 s, 25 cycles of 94° C. for 2 min, 60° C. for 30 s, 72° C. for 30 s followed by a final 5 min extension at 72° C.

The reactions were verified on a 1.5% agarose gel by ethidium bromide staining and the sequences were confirmed by DNA sequence analysis. The gel extracted products were then cloned into pcDNA3.1 Myc/H is (Invitrogen, USA) according to their restriction sites. The G construct was prepared carrying a flag tag at its C-terminus.

Transient Transfection and Cell Growth Suppression:

For cell viability assays, HT-1080 cells were seeded in 24-well tissue culture plate in triplicate 24 h before transfection. At 60-70% confluence, a transfection was performed with 2 μg of pcDNA3.1 (vector control), p16 and its four truncated forms using FuGENE® 6 transfection reagent (Roche, Germany) according to the manufacturer's instructions. 24-48 h after transfection, the cells were harvested and counted using trypan blue dye exclusion. Each assay was completed in triplicate and the results of three independent experiments were reported.

RT-PCR Analysis:

Total RNA was extracted from HT-1080 cells using RNeasy mini kit (Qiagen, Germany) or Tripure reagent (Roche, Germany) 24 h after transfection. 1 μg of RNA was reverse-transcribed using M-MLV Reverse Transcriptase (Fermentas, Ukraine). An expression analysis of p16 and the four truncated constructs was performed in a 25 μl reaction mixture using 1 μl cDNA, 10 pmol of each primer, 0.2 mM dNTP, 1.5 mM MgCl₂, 0.5 U Taq polymerase, 1×PCR buffer and 1% Q-solution (Qiagen, Germany). A PCR amplification was performed under the following conditions: 20 cycles at 94° C. for 30 s, 65° C.-0.5° C. per cycle for 45 s, 72° C. for 30 s, 20 cycles at 94° C. for 2 min, 55° C. for 30 s, 72° C. for 30 s followed by a final 5 min extension at 72° C. β-actin was used as the internal control and amplified with the same condition.

Cell Cycle Analysis:

At 24 h after the transfection of the constructs including the pcDNA3.1 an empty vector, HT-1080 cells was tripsinized and washed once with PBS and re-suspended in hypotonic fluorochrome solution containing 50 μg/ml propidium iodide DNA staining buffer (Sigma, Germany). After incubation for 3 h at 4° C., the cell cycle distribution was recorded in FL3 using fluorescence-activated cell-sorting (FACS; Becton Dickinson, Heidelberg, Germany).

Immunoblotting:

Cellular protein extract was prepared 24-48 h after transfection by re-suspending the cell pellets in lysis buffer containing 30 mM Tris-HCl (pH 7.4), 150 mM NaCl, 1% Triton X-100, 10% glycerol, 1 μM sodium orthovanadate, 5 mM sodium fluoride, 1 mM β-glycerophosphate including 2 mM dithiothreitol (DTT), 200 μM PMSF and 1× protease inhibitor cocktail (Roche, Germany). The cell extracts were separated on 12-17% SDS-polyacrylamide gel electrophoresis and blotted using nitrocellulose membrane (Amersham, Germany). The membranes were then blocked for 1 h in 5% non-fat dried milk in PBS and 1% Tween 20 at room temperature and probed with primary antibodies for 16-20 h at 4° C. as follows: Flag M₂ (F3165, Sigma), Myc (C3956, Sigma), human p53 (610183, BD Transduction), Mcl-1 (AAP-240, Stressgen), Bcl2 (51-6511 GR, PahrMingen), Bim (2819, Cell Signaling), Puma (p 4743, Sigma), Noxa (114C307-1, Alexis), p21 (556431, Pharmingen). After washing the blots with PBST for three times, the bands were visualized using horseradish peroxidase conjugated secondary antibody (Santa Cruz, Germany) and the ECL chemiluminescence detection system (Amersham Bioscience, Germany).

Immunoprecipitation:

At 24-48 h after the transfection, the cells were lysed in ELB (50 mM HEPES), pH 7.0, 0.1% Nonidet p-40, 250 mM NaCl, 5 mM EDTA, 2 mM DTT and 1× protease inhibitor cocktail (Roche, Germany) for 10 min on ice and clarified by centrifugation at 14000×g for 10 min. 1 mg of protein extract was immuno-precipitated with 2 μl of Flag M₂ antibody (F3165, Sigma, Germany) at 4° C. over night followed by capturing with Dynabeads pan mouse IgG (Invitrogen, Germany) for 2 h at 4° C. All immunoprecipitations were washed three times in ELB prior to further manipulation. The labeled proteins were resolved on SDS polyacrylamide gels and subjected to primary antibodies against CDK4 (sc-260, Santa Cruz) and CDK6 (sc-7961, Santa Cruz). An immunodetection was carried out as described above.

Statistical Analysis:

The Data on cell proliferation were analyzed by one-way ANOVA followed by Tukey post-test and p≦0.05 was considered statistically significant. All the experiments were performed in triplicate and the results of the three independent experiments were reported as Mean±SE.

### Results

Effects of p16 Full Length and the Four Truncated Forms on Cell Growth and Colony Formation in HT-1080 Cells:

Following a transfection optimization, p16 and the four truncated forms were introduced into HT-1080 cells. The obtained results indicated that full length p16 can remarkably reduce the cell counts (44.6%) at 24 h which was consistent until 48 h compared with the mock group (vector transfected cells).

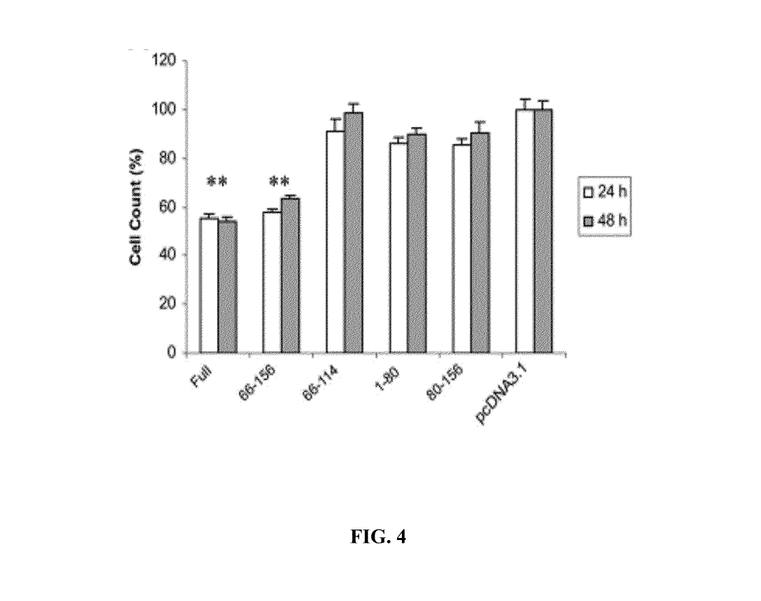

FIG. 4 shows a graphical representation of the effect of p16 and its four truncated forms on the growth and viability of HT-1080 cell line. The cells were transfected with full length p16 and the truncated forms. A cell proliferation was determined by a live cell count using trypan blue. The data were calculated as percent control compared with the vector transfected (pcDNA3.1) group. With respect to FIG. 4, the results from the three independent experiments were presented as Mean±SE, wherein n=3, *p<0.05, **p<0.01. Among the four truncated forms, p16⁶⁶⁻¹⁵⁶ expression demonstrated the comparable results to that of full length p16 and 42% growth inhibition at 24 h compared to the control group, P<0.01. These results show that p16⁶⁶⁻¹⁵⁶ truncated form similar to p16 can act as an inhibitor of cell growth.

To further examine the capability of p16 and the truncated forms on arresting the growth of p16 negative HT-1080 cells, a colony forming assay was performed. The cells were transfected with the five aforementioned plasmids, plated at 5×10³ in 6-well culture dishes and allowed to develop colonies in the presence of G418 for two weeks.

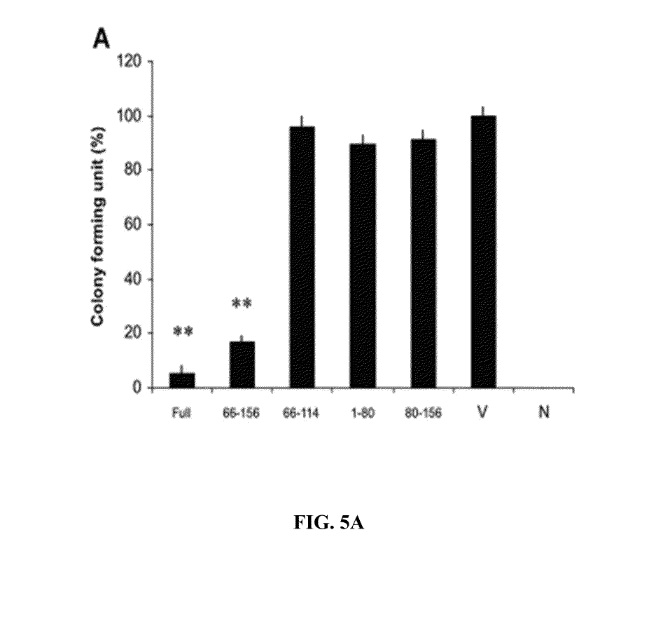

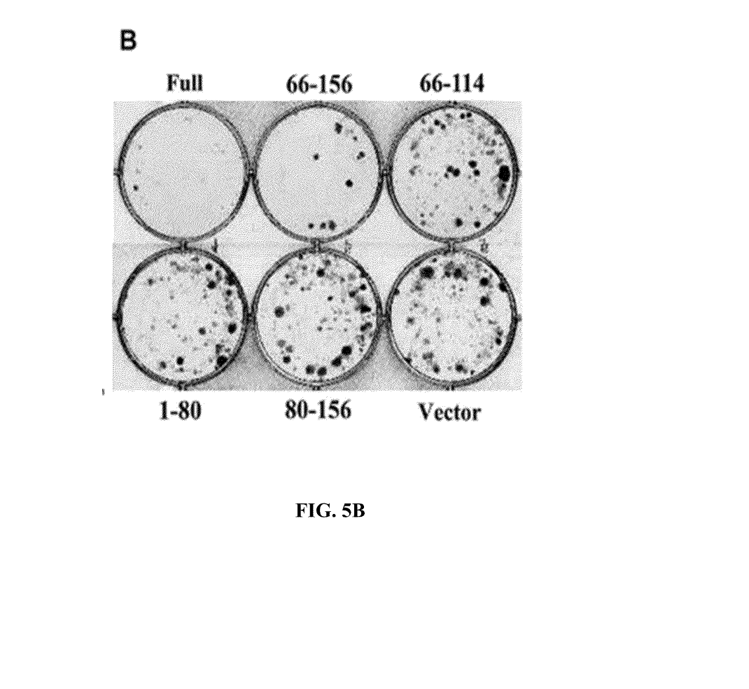

FIG. 5A shows a graphical representation of the effect of p16 and the truncated forms on colony formation in HT-1080 cells. The cells were transfected with p16, B, E, G, H and an empty vector and the resistant colonies were selected with 1 mg/ml G418 for two weeks. With respect to FIG. 5A, the data is presented as mean±SD of percent colony formation relative to pcDNA3.1 based on three independent experiments, wherein n=3.

FIG. 5B shows a top view of the petridishes showing the growth of colonies when stained with crystal violet. With respect to FIG. 5A and FIG. 5B, the resultant number of surviving colonies in p16 and G truncated form transfected cells was significantly less than that of pcDNA3.1 with P=0.0001. These data suggest that this truncated form can be a potent regulator of cell growth like the p16 full length.

Expression of p16 and the Truncated Forms by RT-PCR and Western Blot Analysis:

Expression of p16 and the four truncated forms at mRNA level were confirmed 24 h after a transfection in HT-1080 cells by RT-PCR as described above. The fragments sizes were as expected. β-actin was used as the internal control. HT-1080 cells were transfected with p16 and the truncated forms and cells were harvested and subjected to expression analyses 24 h after transfection.

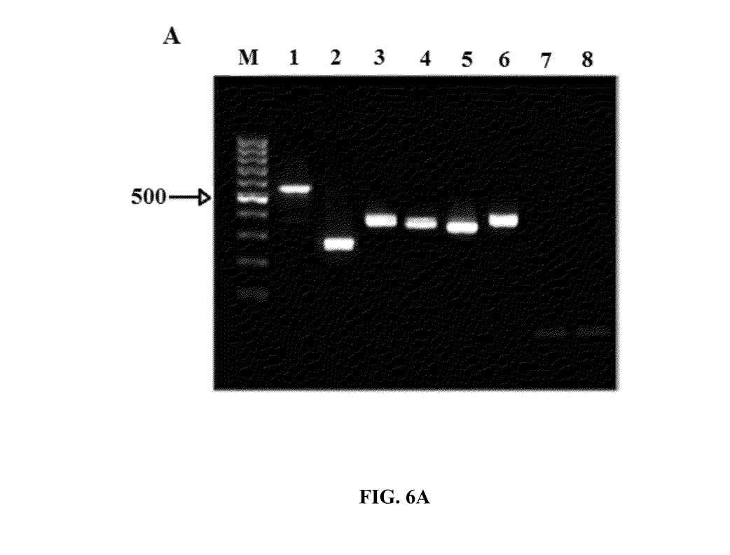

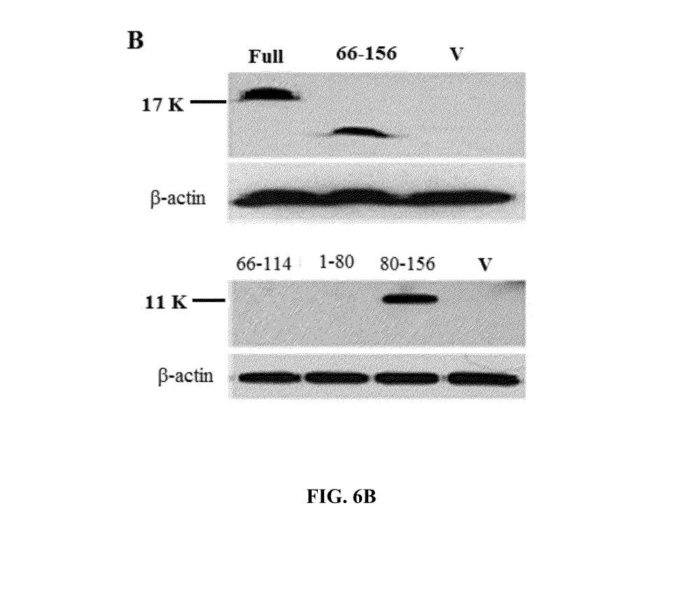

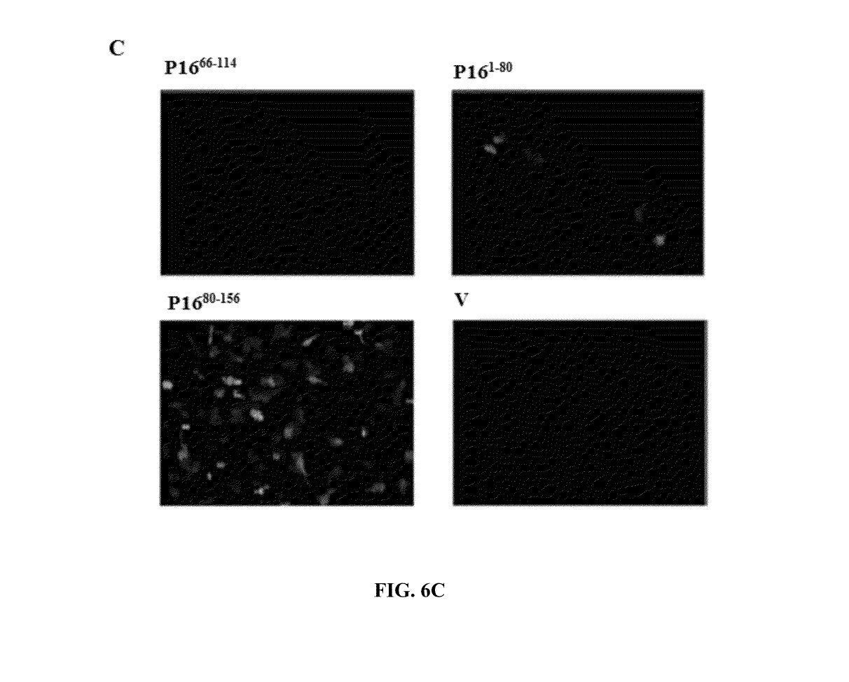

FIG. 6A shows an mRNA expression analysis of p16 and the various truncated forms. The mRNA expression was evaluated by RT-PCR analysis. With respect to FIG. 6A, the lane 1 shows the bands for p16 wild type (lane 1), lane 2 shows the bands for p16⁶⁶⁻¹¹⁴ truncated form (lane 2), lane 3 shows the bands for p16¹⁻⁸⁰ truncated form (lane 3), lane 4 shows the bands for p16⁸⁰⁻¹⁵⁶ truncated form (lane 4), lane 5 shows the bands for p16⁶⁶⁻¹⁵⁶ truncated form (lane 5), lane 6 shows the bands for β-actin (lane 6) and lane 7 shows the bands for pcDNA3.1 empty vector. β-actin was used as the internal control.

FIG. 6B shows a protein expression analysis when evaluated by an immunoblot analysis in the cells transfected with p16 and the various truncated forms. With respect to FIG. 6B, the protein expression evaluated by an immunoblot analysis in the cells transfected with p16 tagged with Flag epitope or p16⁶⁶⁻¹⁵⁶ truncated from tagged with Flag epitope, p16¹⁻⁸° tagged with myc epitope, p16⁶⁶⁻¹¹⁴ tagged with myc epitope, p16⁸⁰⁻¹⁵⁶ truncated form tagged with myc epitope, using Flag and myc antibody respectively. An empty vector transfected cells i.e. pcDNA3.1 were used as the negative control. A protein expression of p16 and the truncated forms were analyzed 24 h after a transfection by immunoblotting. As demonstrated above, the cells transfected with p16, p16⁶⁶⁻¹⁵⁶ and p16⁸⁰⁻¹⁵⁶ construct are expressing high level of protein. However, no expression was detected for p16¹⁻⁸⁰ and p16⁶⁶⁻¹¹⁴ forms expression after transfection.

FIG. 6C shows an immuno fluorescent staining of the transfected cells. With respect to FIG. 6C, the Immuno fluorescent staining of HT-1080 cells after transfection with p16 truncated forms compared to pcDNA3.1 as vector control (V) with anti-Myc monoclonal antibody showed a positive expression of p16⁸⁰⁻¹⁵⁶ and a weak expression of p16¹⁻⁸⁰ truncated form whereas no signal was detected in p16⁶⁶⁻¹¹⁴ transfected cells. The p16¹⁻⁸° construct expression by the immunofluorescent staining in the transfected cells was verified. The p16⁶⁶⁻¹¹⁴ truncated form could not be detected at protein level despite the presence of its mRNA confirmed by RT-PCR analysis.

Cell Cycle Analysis Following Ectopic Expression of p16 and its Four Truncated Forms:

To examine the effect of p16 and the truncated forms expression on cell cycle entry, HT-1080 transfected cells were harvested 24 h past/after transfection and subjected to FACS measurement. A flow cytometric analysis revealed that p16 expression can result in a significant accumulation of the cells in G₀/G₁ phase and decreased a cell population in S phase relative to the vector control. Table 2 shows the Cell cycle distribution after p16 and the four truncated forms of an expression.

Table 2 showing the data for cell cycle distribution after p16 and the four truncated forms expression

| | Vector | p16 | p16⁶⁶⁻¹¹⁴ | p16¹⁻⁸⁰ | p16⁸⁰⁻¹⁵⁶ | p16⁶⁶⁻¹⁵⁶ |
|---|---|---|---|---|---|---|
| G₀/G₁ | 48.64 ± 3.3 | 70.58 ± 1.3* | 45.5 ± 3.1 | 47.97 ± 3.6 | 54.29 ± 3.3 | 62.14 ± 2.1* |
| S | 31.27 ± 4.6 | 14.78 ± 4.1* | 28.8 ± 5.0 | 28.34 ± 4.0 | 24.28 ± 0.6 | 21.95 ± 2.6* |
| G₂ | 18.42 ± 5.9 | 14.33 ± 3.6 | 25.2 ± 1.3 | 23.07 ± 5.4 | 19.63 ± 4.3 | 15.02 2.8 |

The related G₁ arrest correlated directly with the expression status of p16, while reduced p16 expression at 72 h led to the restoring of the cell cycle profile. Interestingly, among the truncated forms, p16⁶⁶⁻¹⁵⁶ construct could function almost similarly to p16 wild type in arresting the G₀/G₁ phase progression in a significant manner at 24 h. None of the other truncated forms altered the cell cycles in transfected cells.

Immunoprecipitation Analysis:

To examine if the binding of p16 or p16⁶⁶⁻¹⁵⁶ to CDK4/6 is the main cause of cell cycle arrest, an immunoprecipitation analysis was performed. In this regard, the cell lysates of HT-1080 cells transfected with p16, p16⁶⁶⁻¹⁵⁶ and pcDNA3.1 plasmids were immunoprecipitated with anti-flag antibody and probed for CDK4 and CDK6 by western blot analysis.

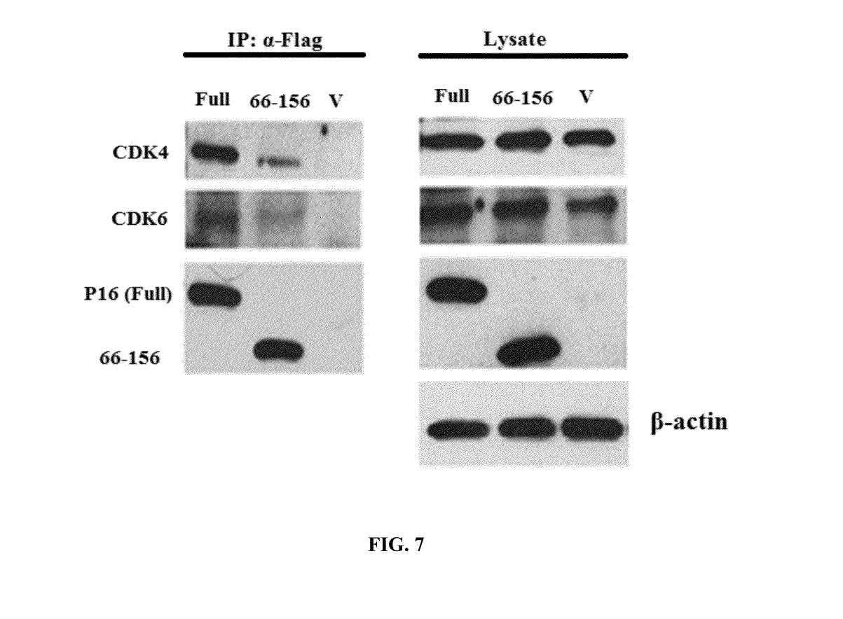

FIG. 7 shows the immunoprecipitation analysis showing the protein interaction of p16 full length and p16⁶⁶⁻¹⁵⁶ truncated form with CDK4 and CDK6. Flag tagged p16 and p16⁶⁶⁻¹⁵⁶ truncated form was transfected into HT-1080 cells and the cell lysate was immunoprecipitated with an anti-flag antibody and probed for CDK4 and CDK6. As indicated a flag tagged p16 and p16⁶⁶⁻¹⁵⁶ truncated from interact with CDK4 and CDK6. With respect to FIG. 7, p16 and p16⁶⁶⁻¹⁵⁶ truncated form interact well with both CDK4 and CDK6, demonstrating that p16⁶⁶⁻¹⁵⁶ construct is capable of binding to CDK4/6 similar to p16 full length.

Effect of p16 and G Truncated Form Ectopic Expression on the Level of pro- and anti-apoptotic proteins:

To address whether an expression of p16 and p16⁶⁶⁻¹⁵⁶ truncated form may have any influence on the level of other proteins involved in proliferation or apoptosis, whole cell lysates of p16 and p16⁶⁶⁻¹⁵⁶ transfected cells were prepared 24 and 48 h after transfection and immunoblotted against Mcl-1, Bcl2, Bim, Puma, Noxa and p21.

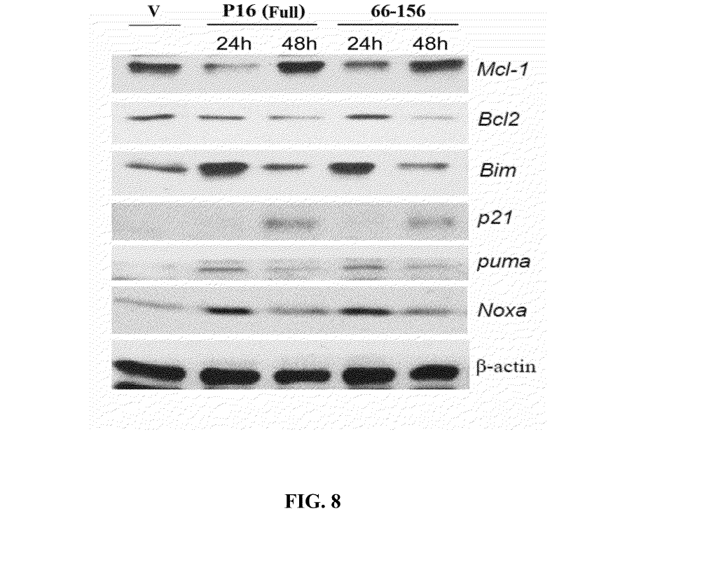

FIG. 8 shows an effect of p16 full length (full) and p16⁶⁶⁻¹⁵⁶ (66-156) truncated from on expression of pro and anti-apoptotic proteins. HT-1080 cells were transfected with p16 and p16⁶⁶⁻¹⁵⁶ truncated form and the cell lysates were prepared at 24 and 48 h and probed for the specific antibodies against Mcl-1, Bcl2, Bim, p21, Puma, and Noxa. β-actin served as a loading control. With respect to FIG. 8, p16 and p16⁶⁶⁻¹⁵⁶ expression were found to down-regulate Mcl-1 at 24 h and Bcl2 at 48 h after a transfection. Moreover, the protein levels of Bim and p21 were clearly up regulated within 24 h and 48 h respectively. There was a slight increase in the amount of Puma and Noxa after 24 h. These results demonstrate that an ectopic expression of p16⁶⁶⁻¹⁵⁶ truncated form induces changes in the cellular protein levels similar to p16 wild type.

To identify the minimum stable and functionally active region of p16, several p16 truncated forms have been generated to be characterized according to their mode of effect on growth inhibition of HT-1080 human fibrosarcoma cells. HT-1080 cells are deficient in p16 expression and positive for Rb and p53 and there has been no previously published data considering the effect of ectopic expression of p16 in this cell line.

Based upon the obtained results, p16⁶⁶⁻¹⁵⁶ truncated form (harboring loop 2, 3, ANKIII, IV and the C-terminal segment) could significantly reduce a cell proliferation and viability, induce a cell cycle arrest and inhibit a colony formation efficiency comparable to p16 wild type. The p16⁶⁶⁻¹⁵⁶ truncated form was able to physically interact with CDK4 and furthermore cause some alterations in the expression of pro and anti-apoptotic proteins such as Noxa, puma, Mcl-1 and Bcl2 similar to the full length p16. It was concluded that the G truncated form can function as good as the full length protein in inhibiting the tumor cell proliferation. The p16⁶⁶⁻¹⁵⁶ embraces all the critical residues for CDK4 interaction like Phe⁹⁰; Glu⁸⁸, Gly⁸⁹, Asp⁹² and Asp⁸⁴ that have roles in a selectivity toward CDK4/6, distortion of the ATP-binding site and binding to CDK4 respectively.

Although outside the interaction sites and not critical for p16 function, the presence of ANKIV as well as the C-terminal part influences the stability and conformation of the remainder part of the fragment G in a way that causes the proper folding and makes it appropriate for CDK4 binding and inhibition. Furthermore, comparing the proliferative inhibitory effects of G and H truncated forms, which are just different in the presence of loop 2, it can be concluded that loop 2 is not only important for CDK4/6 interaction, but also critical for a cell cycle and proliferation inhibition, the features which are absent in H truncated form.

Along with loop 2, a truncated structure G embraces other critical residues for CDK4 interaction like Phe⁹⁰, Glu⁸⁸, Gly⁸⁹ Asp⁹² and Asp⁸⁴ that have roles in a selectivity toward CDK4/6, distortion of the ATP-binding site and binding to CDK4 respectively. Moreover, the truncated form includes the highest frequency regions considering the inactivating mutations corresponding to residues 71-76, 80-102 and 107-127.

Upon the in silico screening analysis followed by the experimental assessments, the novel minimum functional domain of p16 was identified. The novel minimum functional domain of p16 is the C-terminal half including ankyrin repeats III, IV and the C-terminal flanking region accompanied by loops 2 and 3. A transfection of the truncated form into HT-1080 human fibrosarcoma cells, lacking endogenous p16, revealed that it is able to inhibit a cell growth and a proliferation equivalent to p16^{INK4a}. The functional analysis showed that the truncated form like p16 can interact with CDK4/6, block the entry into S phase of the cell cycle and suppress the growth as indicated by the colony formation assay. The identification of p16 minimum functional domain can be of benefit to the future peptidomimetic drug design as well as gene transfer for cancer therapy.

The invention herein reported the identification and a functional characterization of a novel minimum functional domain of p16, possessing amino acids 66-156 which behaves indiscernible from wild type p16 in a cell cycle arrest, a cell growth inhibition and CDK interaction. This functional domain can serve as a template for a peptidomimetic drug design or used as a therapeutic agent to block a cell proliferation in the cancer cells.

A genetic analysis has revealed that certain proteins involved in G₁-S transition state of the cell cycle in particular the cyclin-dependent kinase (CDK)-cyclin D/INK4/pRb/E2F cascade have most often been altered in the human tumors. The tumor suppressor p16INK4a, negative regulator of CDK4/6 and thus G₁-S progression, is one of the most vulnerable genes to the inactivating mutations. This feature along with the various known functional properties of p16 aiming toward a cancer repression has made it a potential candidate from a drug discovery point of view.

In this regard, the designed G truncated form can act as a functional and stable fragment of p16 and can be used to inhibit a cell growth and a proliferation equivalent to p16^{INK4a}. The functional analysis showed that this fragment like p16 can interact with CDK4/6, block the entry into S phase of the cell cycle and suppress growth. Other known functional properties of p16 (i.e. cell senescence, angiogenesis, tumor invasion, matrix-dependent cell spreading, triggering apoptosis and anoikis) can also be evaluated for this functional fragment.

This truncated form can be used for a gene therapy of cancers having non-functional p16 due to the various inactivation reasons such as mutations, deletions or epigenetic silencing.

Additionally, the functional G fragment would serve as a template for drug discovery or be used as a template for a peptidomimetic drug design.

This p16 derived peptide can be used alone or in combination with DNA damaging agents like chemotherapy or radiotherapy to increase the efficacy and decrease the adverse affects.

Few attempts have been made to explore p16 functional domains and examine whether different truncations have any effect on the properties of the protein. The embodiments herein explain a method of designing a stable and functional fragment of p16 tumor suppressor. The embodiments herein shows that a truncated form of p16 including ankyrin repeats III and IV together with loops 2 and 3 can be expressed properly and fold independently. Furthermore, the embodiments herein provide a sufficient data implying that the presence of loop 2, 3 and ANK III, IV as well as the C-terminal segment is sufficient for inducing a cell cycle arrest, a cell growth and a colony forming inhibition. The embodiments herein provide an enough and a comprehensive information to consider almost all the possible truncated forms of p16 to assess their expression, a stability and a mode of action. In this regard, the results show that single ankyrin repeat-containing fragment (fragment E which structurally is made of ANKIII) is not able to be expressed possibly due to the lack of stability. Furthermore, the effectiveness of fragment G in inhibiting a cell proliferation and inducing a cell cycle arrest may strengthen this assumption that the presence of ANKIV as well as the C-terminal part, although outside the interaction sites and not critical for p16 function may influence the stability and conformation of the remainder part of fragment G in a way that causes the proper folding and makes it appropriate for CDK4 binding and inhibition, the feature of which is absent in ANKIII only containing fragment (truncated form E).

In the embodiments herein, HT-1080 human fibrosarcoma cells which are deficient in p16 expression and positive for Rb and p53 are targeted for p16 or other truncated forms functional assessments. There are no previously published data considering the effect of ectopic expression of p16 in this cell line and as there are only few chemotherapy agents that have been considered active in the soft tissue sarcomas, exploiting the gene therapy protocols considering the defective pathways in these cells such as p16/pRb pathway can be rather beneficial toward the better and more specific treatment.

Based upon the results, the enforced expression of p16 in HT-1080 cells significantly reduced the cell proliferation and a viability, induced cell cycle arrest and inhibited colony forming efficiency. Intriguingly, among the evaluated truncated forms, G construct, could act comparable to p16 full length in the mentioned assessments.

It is showed that expression of G truncated form similar to the full length p16 was capable of inducing alteration in the expression of apoptotic and cell cycle involved proteins such as Mcl-1, Bcl2, p21, Bim, Puma and Noxa. These findings suggest that G truncated form can function as good as the full length protein in inhibiting tumor cell proliferation.

Altogether, the embodiments herein report the design and functional characterization of a novel minimum functional p16-derived peptide, containing amino acids 66-156 which behaves indiscernible from a wild type p16 in a cell cycle arrest, a ell growth inhibition and CDK interaction. The functional peptide according to the embodiment herein can serve as a template for drug discovery or used as a therapeutic agent to block a cell proliferation in the cancer cells.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments.

It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A cell growth inhibitor derived from p16^{INK4a} gene comprising:
a C-terminal segment of the p16^{INK4a} gene;
at least two ankyrin repeats, wherein the at least two ankyrin repeats are ankyrin III and ankyrin IV of the p16^{INK4a} gene;
at least two loops, wherein the loops are loop 2 and loop 3 of the p16^{INK4a} gene;
a plurality of residues along with the loop 2, wherein the plurality of residues include Phe⁹⁰, Glu⁸⁸, Gly⁸⁹ Asp⁹² and Asp⁸⁴, and wherein the plurality of residues interact and bind with Cyclin-dependent kinase 4 (CDK4) enzyme, and wherein the plurality of residues have a selectivity towards cyclin-dependent kinase 4/6 (CDK4/6) enzyme, and wherein the plurality of residues cause distortion of ATP-binding sites of a cell.

2. The cell growth inhibitor according to claim 1, wherein the cell growth inhibitor has a gene sequence, wherein the gene sequence is SEQ ID NO. 1.

3. The cell growth inhibitor according to claim 1, wherein the cell growth inhibitor is a truncated form of the p16^{INK4a} gene.

4. The cell growth inhibitor according to claim 1, wherein the cell growth inhibitor encompasses 66-156 amino acids of a full length p16^{INK4a} gene.

5. The cell growth inhibitor according to claim 1, wherein the cell growth inhibitor includes highest frequency regions considering the inactivating mutations corresponding to the residues 71-76, 80-102 and 107-127.

6. The cell growth inhibitor according to claim 1, wherein the cell growth inhibitor has 42% growth inhibition at 24 hrs.

7. A method for synthesizing a cell growth inhibitor comprises:
generating a truncated form, wherein the truncated form is generated by amplifying an expression constructs containing p16 encoding regions and regulatory elements necessary for an expression of a transcript using a Polymeric Chain Reaction (PCR) using a specific primer pairs carrying restriction enzyme sites; and
cloning the generated truncated form into pcDNA3.1 mammalian expression vector.

8. The method according to claim 7, wherein the specific primer pairs are a forward and a reverse primer, and wherein the forward primer is GAGGAATTCACCATGCACGGCGC (SEQ ID NO: 2) and wherein the reverse primer is TATGCGGCCGCTCACTTGTCGTC (SEQ ID NO: 3). The method according to claim 7, wherein the cloning is done on a cloning site, wherein the cloning site are EcoRI and NotI.

9. The method according to claim 9, wherein the EcoRI is a cloning site for a forward primer.

10. The method according to claim 9, wherein the NotI is a cloning site for a reverse primer.

11. The method according to claim 7, wherein the cell growth inhibitor is a truncated form of the p16^{INK4a} gene.

12. The method according to claim 7, wherein the cell growth inhibitor encompasses 66-156 amino acids of a full length p16^{INK4a} gene.

13. The method according to claim 7, wherein the cell growth inhibitor has 42% growth inhibition at 24 hrs.

14. A cell growth inhibitor structure comprising a truncated form (p16⁶⁶⁻¹⁵⁶) encompassing amino acids 66 to 156 of a full length p16 gene, wherein the truncated form includes a C-terminal segment of a p16 gene comprising ankyrin repeats III and IV along with loops 2 and 3 of p16 gene.


SEQUENCE LISTING (9 sequences)

SEQ ID NO: 1 - DNA, 324 bp, Artificial Sequence
  Synthetic construct cell growth inhibitor
aacatgcacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggccgaggagctgggccatcgcgatgtcgcacggtacctgcgcgcggctgcggggggcaccagaggcagtaaccatgcccgcatagatgccgcggaaggtccctcagacatccccgattacaaagtggttgctagcgactacaaagacgatgacgacaagtga

SEQ ID NO: 2 - DNA, 23 bp, Artificial Sequence
  Synthetic primer
gaggaattcaccatgcacggcgc

SEQ ID NO: 3 - DNA, 23 bp, Artificial Sequence
  Synthetic primer
tatgcggccgctcacttgtcgtc

SEQ ID NO: 4 - DNA, 27 bp, Artificial Sequence
  Synthetic primer
ttgaaggaattcggtaccatggagccg

SEQ ID NO: 5 - DNA, 26 bp, Artificial Sequence
  Synthetic primer
cgtcgtgctcgagtcgggtgagagtg

SEQ ID NO: 6 - DNA, 23 bp, Artificial Sequence
  Synthetic primer
ggcggagaagcttgcgatgcacg

SEQ ID NO: 7 - DNA, 21 bp, Artificial Sequence
  Synthetic primer
ccagctcgaggggcagacggc

SEQ ID NO: 8 - DNA, 26 bp, Artificial Sequence
  Synthetic primer
cgccaagcttaccatgcgacccgtgc

SEQ ID NO: 9 - DNA, 26 bp, Artificial Sequence
  Synthetic primer
cctctagaatcggggatgtctgaggg